(19) United States
(12) Patent Application Publication
(10) Pub. No.: US 2012/0107363 A1
(43) Pub. Date: May 3, 2012

(54) IMMUNOGEN AGAINST CAMPYLOBACTER INFECTION

(76) Inventor: Patricia Guery-Kopecko, Silver Spring, MD (US)

(21) Appl. No.: 13/343,153

(22) Filed: Jan. 4, 2012

Related U.S. Application Data
(62) Division of application No. 12/874,304, filed on Sep. 2, 2010, now abandoned.
(60) Provisional application No. 61/239,969, filed on Sep. 4, 2009.

(51) Int. Cl. A61K 39/05; A61P 31/04; C07K 14/205

(52) U.S. Cl. 424/245.1; 530/350

(57) ABSTRACT

The invention relates to the use of Campylobacter jejuni gene product jlpA and immunogenic fragments of jlpA as a component of a pharmaceutical formulation capable of eliciting an anti-Campylobacter jejuni immune response. The invention also relates to a method of inducing an anti-Campylobacter immune response by the administration of C. jejuni jlpA gene products or immunogenic fragments of such gene product to a subject.

### CROSS-REFERENCE TO RELATED APPLICATION

This application claims priority to Provisional Application No. 61/239,969 filed Sep. 4, 2009 (now expired), and a divisional application of U.S. patent application Ser. No. 12/874,304 filed on 2 Sep. 2010.

### TECHNICAL FIELD

The invention relates to a Campylobacter jejuni gene product and immunogenic fragments of the gene product useful as component(s) of a pharmaceutical formulation capable of eliciting an anti-Campylobacter jejuni immune response. The invention also relates to a method of inducing an anti-Campylobacter immune response by the administration of C. jejuni jlpA gene products or immunogenic fragments of such gene product.

Campylobacter jejuni is a Gram-negative, spiral, micro-aerophilic bacterium that exists as a commensal organism in the intestinal tracts of a variety of wild and domestic animals. It is a leading cause of acute bacterial enterocolitis in humans. Clinical symptoms of campylobacteriosis range from a mild watery diarrhea to bloody diarrhea accompanied by nausea, vomiting, fever, abdominal cramps and the presence of fecal leucocytes.

Campylobacter infection also has been associated with Guillain-Barré syndrome, an autoimmune-mediated disorder of the peripheral nervous system causing paralysis and even death. Like many other human enteric bacterial pathogens, C. jejuni infects humans by colonizing the mucus layer of the intestine followed by adherence and invasion of epithelial cells.

Although Campylobacter infection is treatable with antibiotics, an effective vaccine formulation against the organism is desired for disease prevention. Vaccine administration is especially beneficial for travelers to regions of the world where Campylobacter is endemic. However, there are currently no licensed vaccines for this organism.

Adherence of C. jejuni is a multifactorial event in which multiple binding factors may be required to bind to their respective receptors to achieve an efficient interaction with host cells. Campylobacter jejuni surface constituents, including PEB1 (Pei et al., 1998), CadF (Konkel et al., 1997), JlpA (Jin et al., 2001 and Jin et al., 2003), a 43 kDa major outer membrane protein (MOMP) (Moser et al., 1997), lipopolysaccharides (LPS) (Fry et al., 2000), and motility provided by the polar flagellum of the bacterium (Yao et al., 1994), each play a role in promoting bacterial adherence. Non-specific binding of the bacterium to epithelial cell lipids may also mediate adherence (Szymanski and Armstrong, 1996).

Jin (2001) identified an 1116 base pair open reading frame (ORF), designated jlpA, encoding a novel species-specific lipoprotein of Campylobacter jejuni TGH9011, from recombinant plasmid pHIP-O. The jlpA gene encodes a polypeptide (hereafter “JlpA”) of 372 amino acid residues with a molecular mass of 42.3 kDa. JlpA contains a typical signal peptide and lipoprotein processing site at the N-terminus. The presence of a lipid moiety on the JlpA molecule was confirmed by the incorporation of [³H]-palmitic acid. Immunoblotting analysis of cell surface extracts indicated that JlpA is a surface-exposed lipoprotein in C. jejuni. JlpA is loosely associated with the cell surface, as it is easily extracted from the C. jejuni outer membrane by detergents. The adherence of both insertion and deletion mutants of jlpA to HEp-2 epithelial cells was reduced compared with that of parental C. jejuni TGH9011. Adherence of C. jejuni to HEp-2 cells was inhibited in a dose-dependent manner when the bacterium was preincubated with anti-GST-JlpA antibodies or when HEp-2 cells were preincubated with JlpA protein. A ligand-binding immunoblotting assay showed that JlpA binds to HEp-2 cells, which suggests that the C. jejuni surface lipoprotein JlpA mediates adherence of the bacterium to epithelial cells.

Jin (2003) further demonstrated that JlpA interacts with HEp-2 cell surface heat shock protein (Hsp) 90α and initiates signaling pathways leading to activation of NF-κB and p38 MAP kinase. Gel overlay and GST pull down assays showed that JlpA interacts with Hsp90α. Geldanamycin, a specific inhibitor of Hsp90, and anti-human Hsp90α antibody significantly blocked the interaction between JlpA and Hsp90α, suggesting a direct interaction between JlpA and HEp-2 cell surface-exposed Hsp90α. The treatment of HEp-2 cells with GST-JlpA initiated two signaling pathways: one leading to the phosphorylation and degradation of IκB and nuclear translocation of NF-κB; and another one to the phosphorylation of p38 MAP kinase. The activation of NF-κB and p38 MAP kinase in HEp-2 cells suggests that JlpA triggers inflammatory/immune responses in host cells following C. jejuni infection.

### BRIEF DESCRIPTION OF THE DRAWINGS

FIG. 1: Alignment of Amino Acid Sequences of JlpA of different Campylobacter strains.

FIG. 2: JLPA-specific serum IgG responses in mice.

FIG. 3: JLPA-specific fecal IgA responses in mice.

### DETAILED DESCRIPTION OF PREFERRED EMBODIMENTS

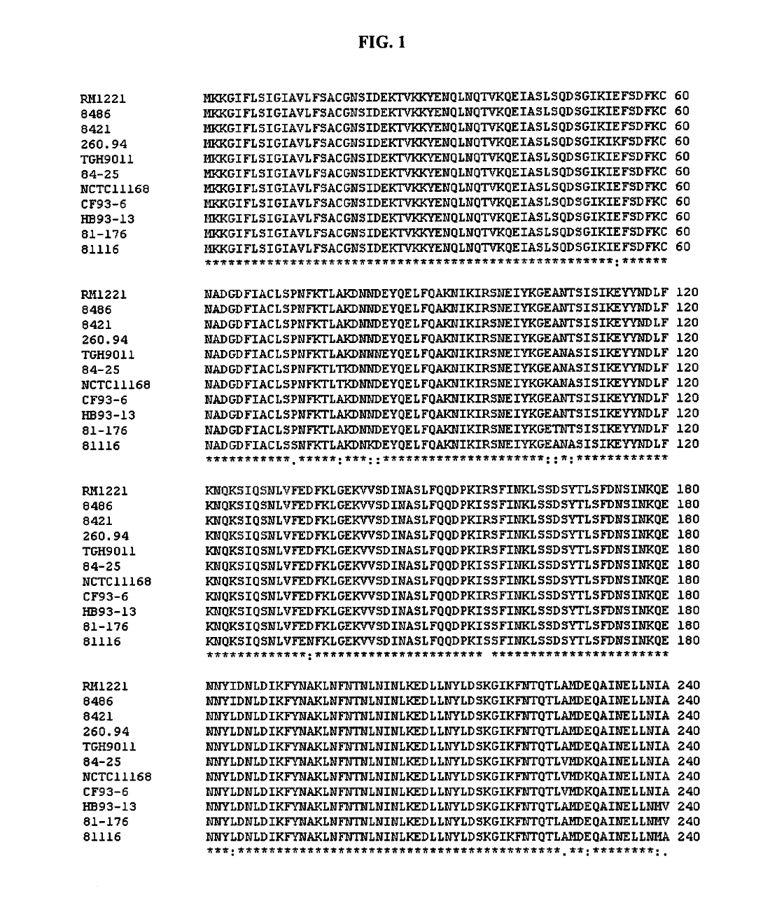

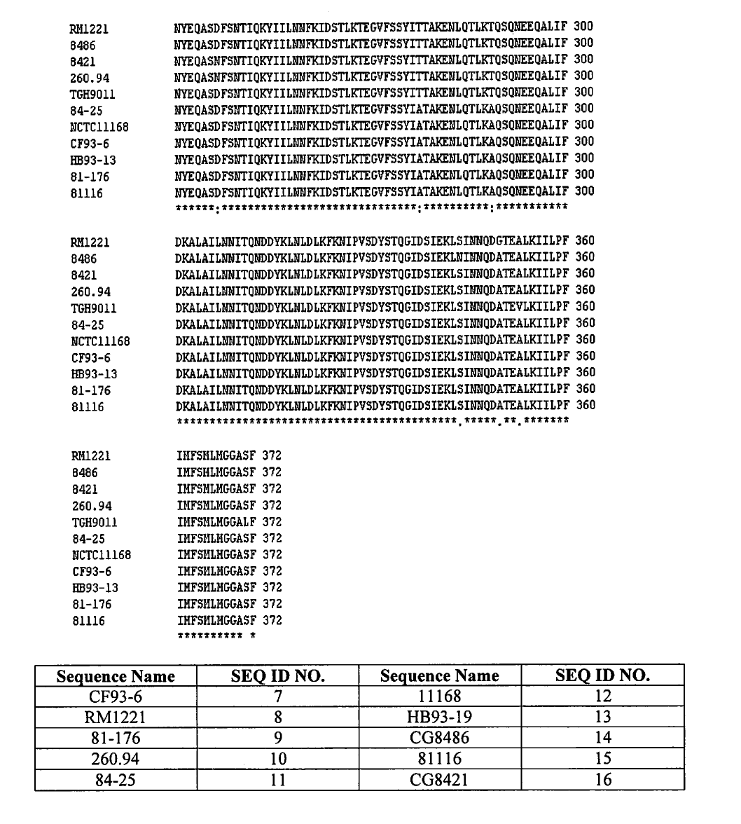

JlpA is a Campylobacter specific protein and is highly conserved among Campylobacter strains (FIG. 1), which make it a valuable vaccine candidate against C. jejuni. An aspect of this invention is an immunogenic composition comprising Campylobacter jejuni protein JlpA or fragments thereof. The composition can be isolated and prepared from natural C. jejuni isolates or be recombinantly produced. Another aspect of this invention is a method of inducing an immune response against Campylobacter by the administration of JlpA or its immunogenic fragments.

### Example 1

### Expression of JlpA as a Recombinant Histidine Tagged Protein in E. coli

In an exemplary embodiment of the invention, the jlpA gene from Campylobacter jejuni 81-176 was amplified by polymerase chain reaction (hereafter “PCR”) with the jlpA forward primer:

(SEQ ID. NO. 5)
CCA TAT GTG CGG AAA TTC CAT AGA TGA AAA AAC;

and jlpA reverse primer:

(SEQ ID. NO. 6)
GGG ATC CTT AAA ATG ACG CTC CGC CC..

The polymerase used for the amplification in this embodiment was HF2 DNA polymerase (Clontech Laboratories, Inc. Mountain View, Calif.). The primers introduced NdeI and BamHI sites into the amplicon. The amplicon was digested with NdeI and BamHI and ligated to NdeI and BamHI-digested pET19b plasmid (NOVAGEN®, Gibbstown, N.J.). The ligation mixture was used to transform DH5 alpha cells with selection on ampicillin resulting in strain PG2604. The DNA in this plasmid was sequenced to confirm that the appropriate gene had been cloned. The plasmid in PG2604 was transformed into BL21 (DE3) to express recombinant JlpA with a hexahistidine tag. The confirmed DNA sequence of the cloned jlpA gene with a hexahistidine tag was identified in SEQ ID. NO. 4. The predicted protein encoded by this DNA sequence had a predicted mass of 43.33 kDa and a predicted pI of 5.03. The predicted protein sequence was identified in SEQ ID. NO. 3.

### Example 2

### Immunogenicity of JIpA During Human Infection with C. jejuni

Study subjects were diarrhea patients enrolled in field studies during Cobra Gold 1999, in Thailand. The pathogens isolated are shown in the Table 1. A group of 10 subjects who participated in the Cobra Gold exercise in 2000 in Thailand, but did not develop diarrhea were used as negative controls.

Plasma samples were collected on study days 7 and 60, following presentation at the clinic. A single blood sample was collected from the control group during the exercise. Samples were kept frozen until assayed for JlpA81-176-specific IgA or IgG using ELISA. Mean+2 standard deviation value of the control group was used to determine responder rates among the infected groups. Mean IgA and IgG titers among the control group were 1,040±1,032 and 3,800±2,437, respectively. Responders were defined as individual having IgA titer of 1:3,200 and IgG titer of ≧1:9,000. Following infection (in a field setting), systemic immune response to JlpA was observed at the time points tested in 50% of the subjects recovering from Campylobacter infection.

TABLE 1
Systemic immune responses to JlpA antigen in humans

| | | Serum responses to JlpA (% responders) | | |
|---|---|---|---|---|
| Pathogen Isolated | N | IgA | IgG | Any Isotype |
| Campylobacter only | 18 | 38.9 | 16.7 | 50.0 |
| Salmonella only | 8 | 0 | 30.0 | 30.0 |
| Other Pathogens | 7 | 0 | 14.3 | 14.3 |
| None | 7 | 0 | 0 | 0 |
| Control | 10 | 0 | 0 | 0 |

### Example 3

### Immunogenicity and Efficacy Of Protein JlpA of C. jejuni Strains in Mice

A murine study were undertaken in order to evaluate the immunogenicity and potential protective efficacy of an embodiment of the vaccine composition. In the study, adult female BALB/c mice were lightly anesthetized with isoflurane and immunized intranasally with 30 μl of PBS containing recombinant JlpA protein alone or with a mucosal adjuvant, LTR192G. The experimental groups are illustrated in Table 2.

TABLE 2
Experimental groups for JLPA vaccination

| | Recombinant | | |
|---|---|---|---|
| Group | JlpA (μg) | LTR192G (μg) | N |
| 1 | 0 | − | 8 |
| 2 | 5 | + | 8 |
| 3 | 25 | − | 7 |
| 4 | 25 | + | 7 |
| 5 | 100 | − | 7 |
| 6 | 100 | + | 7 |

Three doses of the embodiment vaccine composition were delivered at 14 day intervals to the subject, on study days: 0, 14 and 28. Seven days after the last vaccination, on study day 35, antigen-specific secretory IgA was measured from fecal pellets. Antigen-specific serum IgG was determined in tail blood collected 21-22 days after the last vaccination, on study day 49-50. All antigen-specific responses were detected using ELISA.

On study day 58 (30 days post last vaccination), all animals were challenged intranasally with 3×10⁹ cfu of C. jejuni 81-176. Following the challenge, animals were followed for 6 consecutive days for the development of the infection associated illness. Based on the severity of sickness a score was assigned to each animal as follows.
0=No apparent illness
1=ruffled fur
2=ruffled fur and hunched back
3=dead
   Daily sickness indices, and then group average indices were determined and vaccine efficacy was calculated as: (Control—vaccinated)/(Control)×100

### Immunogenicity of JlpA

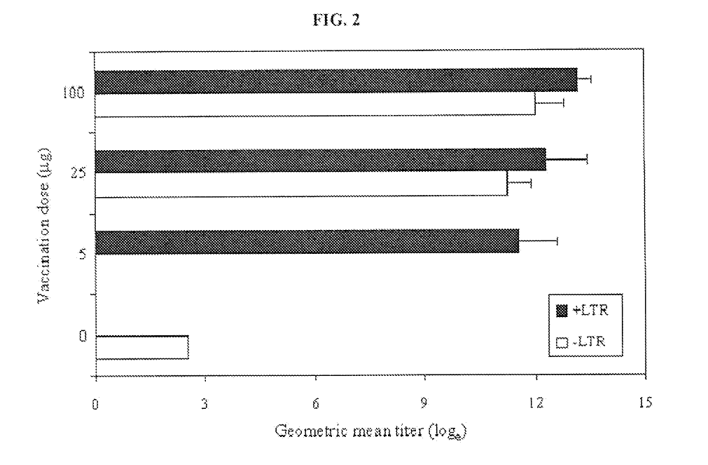

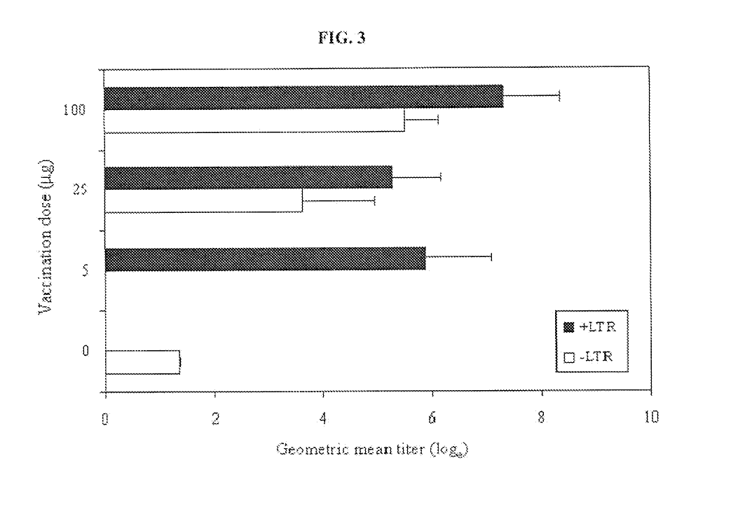

JlpA-specific serum IgG responses in mice were shown in FIG. 2. JlpA-specific fecal IgA responses in mice were shown in FIG. 3. Data are presented as group geometric mean titer (natural logₑ) and standard deviation.

As illustrated in FIG. 2, a robust vaccine dose dependent serum IgG levels were detected in animals immunized with recombinant JlpA alone, which was further enhanced by inclusion of a mucosal adjuvant LTR192G (FIG. 2). Animals immunized with PBS showed low levels of anti-JlpA IgG responses (mean+2 standard deviations≦1:100). Based on this cut off, all animals in all groups immunized with or without LTR192G were categorized as IgG responders.

Similar to serum IgG response, a robust mucosal IgA response was detected after vaccination. Interestingly, LTR192G had a greater adjuvant effect on mucosal IgA response than observed for serum IgG response, with 5 to 6 fold increase when the same amount of protein was delivered with the adjuvant. Animals immunized with PBS showed no detectable levels anti-JlpA fecal IgA response (mean+2 standard deviations≦1:4). Based on this cut off, 100% of the animals receiving 100 μg of vaccine alone or vaccine doses tested (5 μg, 25 μg, 100 μg) with the adjuvant were categorized as responders for IgA. Five of 6 animals (83%; sample was unavailable for one animal) receiving 25 μg showed detectable levels of JlpA-specific IgA in stool extracts.

### Protective Efficacy of JlpA

Following the challenge with C. jejuni 81-176, animals were observed for the development of sickness. The illness index and calculated JlpA efficacy of different vaccine formulations are presented Table 3.

TABLE 3
JlpA efficacy in mice challenged with C. jejuni 81-176

| | Recombinant | | Illness index | |
|---|---|---|---|---|
| Group | JlpA (μg) | LTR192G (μg) | (mean ± sd) | Efficacy ((%) |
| 1 | 0 | − | 1.21 ± 0.30 | n/a |
| 2 | 5 | − | nd | |
| 3 | 5 | + | 1.23 ± 0.31 | −6.8 |
| 4 | 25 | − | 1.01 ± 0.24 | 16.1 |
| 5 | 25 | + | 0.78 ± 0.21 | 35.4 |
| 6 | 100 | − | 0.77 ± 0.54 | 35.6 |
| 7 | 100 | + | 0.41 ± 0.25 | 66.3 |

Twenty four hours following each vaccination, animals receiving 25 μg or higher dose of vaccine with the adjuvant showed a mild transient signs of ruffled fur, which lasted for <24 hours. No apparent signs of vaccine associated side effects were seen among animals receiving PBS or vaccine alone. Animals immunized with 100 μg of the vaccine with the adjuvant showed 66.3% protection. To achieve a moderate (approximately 35%) protection, a 100 μg dose alone or 25 μg with the adjuvant was required. Other doses showed no protection against illness in mice.

These studies illustrate the utility of the JlpA construct of C. jejuni 81-176 as components, alone or in combination with other moieties, as vaccines against Campylobacter. Accordingly, the inventive constructs can be used in methods for induction of protective anti-Campylobacter immunity

### Prophetic Example

### Induction of the Anti-C. jejuni Mediated Response in Human

A prophetic method for the induction of the anti-C. jejuni mediated response in humans contains the following steps:
a. administration of immunogen comprising JlpA, or immunogenic fragments thereof, with or without a tag, such as histidine. If a boosting dose or doses are to be given this first administration of immunogen is a priming dose. The immunogen can be derived from isolated native polypeptide or recombinantly produced JlpA, or JlpA fragments. The immunogen can be administered orally, nasally, subcutaneously, intradermally, transdermally, transcutaneously intramuscularly, or rectally. The range of a unit dose of immunogen is 25 μg to 1 mg of immunogen. The immunogen is administered in any number of aqueous buffered solutions with or without carrier protein or adjuvant. The adjuvant can be any number of potential adjuvants, including but not limited to LTR 192G, Aluminum hydroxide, RC529E, QS21, E294, oligodeoxynucleotides (ODN), CpG-containing oligodeoxynucleotides, aluminum phosphate, MPL® (GlaxoSmithKline, Middlesex, UK) or combinations of these or other potential adjuvants.
b. The inventive method also contemplates immunization with or without administration of subsequent boosting. The boosting step comprises the administration of, subsequent to a priming dose, 1 to 4 boosting doses with a unit dose range of 50 μg to 1 mg of immunogen in buffered aqueous solutions.

Alternative embodiments of the inventive method include inserting nucleotide constructs encoding JlpA, or fragments thereof, into an expression system capable of expression in mammalian subjects. In this embodiment, the expression system can be a plasmid, viral or DNA expression vector. Another alternative embodiment of the inventive method is to induce immunity by administering a live attenuated carrier strain of bacteria transformed with a suitable viral or DNA expression system that contains one or more nucleic acid sequences encoding one or more of the polypeptides JlpA, or fragments thereof. The expression system contemplated is capable of expressing in the selected bacteria carrier.

Having described the invention, one skilled in the art will appreciate in the appended claims that many modifications and variations of the present invention are possible in light of the above teaching. It is therefore, to be understood that, within the scope of the appended claims, the invention may be practiced otherwise than as specifically described.

### REFERENCES

Fry, B. N., Feng, S., Chen, Y., Newell, D. G., Coloe, P. J., and Korolik, V. (2000) The galE gene of Campylobacter jejuni is involved in lipopolysaccharide synthesis and virulence. Infect Immun 68: 2594-2601.
Jin, S., Joe, A., Lynett, J., Hani, E., Sherman, P., and Chan, V. L. (2001) JlpA, a novel surface-exposed lipoprotein specific to Campylobacter jejuni, mediates adherence to host epithelial cells. Mol Microbiol 39: 1225-1236.
Jin S., Song Y. C., Emili A., Sherman P. M. and Chan V L (2003) JlpA of Campylobacter jejuni interacts with surface-exposed heat shock protein 90α and triggers signalling pathways leading to the activation of NF-κB and p38 MAP kinase in epithelial cells.
Konkel, M. E., Garvis, S. G., Tipton, S. L., Anderson, D. E. Cieplak, W., Jr (1997) Identification and molecular cloning of a gene encoding a fibronectin-binding protein (CadF) from Campylobacter jejuni. Mol Microbiol 24: 953-963.
Moser, I., Schroeder, W., and Salnikow, J. (1997) Campylobacter jejuni major outer membrane protein and a 59-kDa protein are involved in binding to fibronectin and INT 407 cell membranes. FEMS Microbiol Lett 157: 233-238.
Pei, Z., Burucoa, C., Grignon, B., Baqar, S., Huang, X., Kopecko, J., et al. (1998) Mutation in the pel1A locus of Campylobacter jejuni reduces interactions with epithelial cells and intestinal colonization of mice. Infect Immun 66: 938-946.
Szymanski, C. M., and Armstrong, G. D. (1996) Interactions between Campylobacter jejuni and lipids. Infect Immun 64: 3467-3474.
Yao, R., Burr, D. H., Doig, P., Trust, T. J., Niu, H., and Guerry, P. (1994) Isolation of motile and non-motile insertional mutants of Campylobacter jejuni: the role of motility in adherence and invasion of eukaryotic cells. Mol Microbiol 14: 883-893.

What is claimed is:

1. An immunogenic composition comprising an isolated protein encoded by jlpA gene of Campylobacter jejuni or a fragment thereof, from one or more strains of Campylobacter jejuni.

2. The immunogenic composition of claim 1, wherein said protein is a recombinant polypeptide with an amino acid sequence set forth in SEQ ID No. 1 or fragments thereof.

3. The immunogenic composition of claim 2, wherein said protein contains a histidine tag.

4. The immunogenic composition of claim 1, wherein said protein is encoded by a nucleic acid sequence set forth in SEQ ID No. 2.

5. The immunogenic composition of claim 1, where said strain of Campylobacter jejuni is selected from the group consisting of 8421, 260.94, RM1221, 8486, TGH 9011, HB93-13, OH4384, 81-176, CF93-6, 84-25, NCTC 11168, and 81116.

6. The immunogenic composition of claim 1, further comprising an adjuvant.

7. The immunogenic composition of claim 6, wherein said adjuvant is selected from the group consisting: LTR192G, Aluminum hydroxide, RC529E, QS21, E294, oligodeoxynucleotides (ODN), CpG-containing oligodeoxynucleotides, aluminum phosphate, MPL® and a combination thereof.

8. A method for inducing an immune response against Campylobacter jejuni, comprising administering a dose of said immunogenic composition of claim 1 to a subject.

9. The method of claim 8, comprising the additional step of administering one or more boosting doses subsequent to said dose wherein said boosting dose is comprised of the same said immunogenic composition as administered in said dose.

10. The method of claim 9, wherein said composition is a polypeptide set forth in SEQ ID No. 1 or fragments thereof.

11. The method of claim 10, wherein said polypeptide contains a histidine tag.

12. The method of claim 8, wherein said dose is administered by a route selected from the group consisting of intranasally, subcutaneously, transdermally, orally and intravenously.

13. The method of claim 9, wherein said boosting doses are administered as in formulation selected from the group consisting of injectable formulation, intranasal formulation, oral formulation, subcutaneous formulation.

14. The method of claim 10, wherein said dose comprising about 25 μg to 1 mg of said polypeptide.

15. The method of claim 8, wherein said protein is encoded by a nucleic acid sequence set forth in SEQ ID. NO. 2.

16. The method of claim 15, wherein said nucleotide sequence is inserted in an expression vector wherein said expression vector is selected from the group consisting of plasmid, viral expression vectors and wherein said expression vector is functional in mammalian subjects and wherein said polypeptide sequence is expressed.

17. The method of claim 16, wherein said nucleotide sequences are inserted in a plasmid or viral expression vector system and expressed in a live, attenuated strain of carrier bacteria.

18. The method of claim 17, wherein said attenuated strain of carrier bacteria is selected from the group consisting of Escherichia coli, member of the genus Shigella, member of the genus Campylobacter, member of the genus Salmonella, member of the genus Vibrio.

19. The method of claim 16, wherein said plasmid vector is selected from the group consisting of pMal, pQE, PRO Tet bacterial expression system, and pET.

20. The method of claim 16, wherein said viral expression vector is selected from the group consisting of adenovirus, M13, herpesvirus, vaccinia virus and baculovirus.

21. A method of reducing campylobacter intestinal colonization in a subject, said method comprising administering an immunogenically effective amount of immunogenic composition of claim 1 with or without an adjuvant.

22. A vaccine against Campylobacter jejuni comprising an isolated protein encoded by jlpA gene of Campylobacter jejuni or a fragment thereof, from one or more strains of Campylobacter jejuni.


SEQUENCE LISTING (16 sequences)

SEQ ID NO: 1 - protein, 349 aa, Campylobacter jejuni
CGNSIDEKTVKKYENQLNQTVKQEIASLSQDSGIKIEFSDFKCNADGDFIACLSPNFKTLAKDNNDEYQELFQAKNIKIRSNEIYKGETNTSISIKEYYNDLFKNQKSIQSNLVFEDFKLGEKVVSDINASLFQQDPKISSFINKLSSDSYTLSFDNSINKQENNYLDNLDIKFYNAKLNFNTNLNINLKEDLLNYLDSKGIKFNTQTLAMDEQAINELLNMVNYEQASDFSNTIQKYIILNNFKIDSTLKTEGVFSSYIATAKENLQTLKAQSQNEEQALIFDKALAILNNITQNDDYKLNLDLKFKNIPVSDYSTQGIDSIEKLSINNQDATEALKIILPFIMFSML

SEQ ID NO: 2 - DNA, 1068 bp, Campylobacter jejuni
tgcggaaattccatagatgaaaaaacagttaaaaaatatgaaaatcaactaaatcaaacagttaaacaagaaattgcaagcctttctcaggattctggaataaagattgaattctcagattttaaatgcaatgcagatggtgattttatagcgtgtttaagtccaaatttcaaaactcttgcaaaagataataacgatgagtatcaagaactatttcaagcaaaaaatatcaaaattcgctcaaatgaaatttacaaaggcgaaacaaatacatcaattagcattaaagaatattacaacgatctttttaaaaaccaaaaaagcatacaatcaaatttggtttttgaagattttaaacttggagaaaaagttgttagtgacatcaatgcaagcttatttcaacaagatccaaaaatcagctcttttatcaataaattaagctcggattcttatactttatcatttgataactccataaacaaacaagaaaacaattatctagataatcttgatataaaattttataacgcaaaattaaatttcaatacaaatctaaatataaatctaaaagaagatttacttaattaccttgattctaagggtataaaatttaacactcaaactttagctatggatgaacaagctatcaatgaacttcttaatatggtaaactacgaacaagcttcagattttagcaatacaatccaaaaatacatcatcttaaataattttaaaattgattcaactcttaaaaccgaaggcgtatttagtagctatatagcaactgctaaggaaaatttacaaaccttaaaagctcaaagtcaaaatgaagaacaagcattaatctttgataaagctttagctattttaaacaatatcactcaaaatgatgattataaactcaatcttgatttaaaatttaaaaacatccctgttagtgactatagcacacagggaatcgacagcatagaaaaactaagcatcaacaatcaagacgccacagaagcgttaaaaattatacttccatttatcatgttttctatgttaatgggcggagcgtcattttaa

SEQ ID NO: 3 - protein, 379 aa, Campylobacter jejuni
MGHHHHHHHHHHSSGHIDDDDKHMCGNSIDEKTVKKYENQLNQTVKQEIASLSQDSGIKIEFSDFKCNADGDFIACLSPNFKTLAKDNNDEYQELFQAKNIKIRSNEIYKGETNTSISIKEYYNDLFKNQKSIQSNLVFEDFKLGEKVVSDINASLFQQDPKISSFINKLSSDSYTLSFDNSINKQENNYLDNLDIKFYNAKLNFNTNLNINLKEDLLNYLDSKGIKFNTQTLAMDEQAINELLNMVNYEQASDFSNTIQKYIILNNFKIDSTLKTEGVFSSYIATAKENLQTLKAQSQNEEQALIFDKALAILNNITQNDDYKLNLDLKFKNIPVSDYSTQGIDSIEKLSINNQDATEALKIILPFIMFSMLMGGASF

SEQ ID NO: 4 - DNA, 1179 bp, Campylobacter jejuni
atgggccatcatcatcatcatcatcatcatcatcacagcagcggccatatcgacgacgacgacaagcatatgtgcggaaattccatagatgaaaaaacagttaaaaaatatgaaaatcaactaaatcaaacagttaaacaagaaattgcaagcctttctcaggattctggaataaagattgaattctcagattttaaatgcaatgcagatggtgattttatagcgtgtttaagtccaaatttcaaaactcttgcaaaagataataacgatgagtatcaagaactatttcaagcaaaaaatatcaaaattcgctcaaatgaaatttacaaaggcgaaacaaatacatcaattagcattaaagaatattacaacgatctttttaaaaaccaaaaaagcatacaatcaaatttggtttttgaagattttaaacttggagaaaaagttgttagtgacatcaatgcaagcttatttcaacaagatccaaaaatcagctcttttatcaataaattaagctcggattcttatactttatcatttgataactccataaacaaacaagaaaacaattatctagataatcttgatataaaattttataacgcaaaattaaatttcaatacaaatctaaatataaatctaaaagaagatttacttaattaccttgattctaagggtataaaatttaacactcaaactttagctatggatgaacaagctatcaatgaacttcttaatatggtaaactacgaacaagcttcagattttagcaatacaatccaaaaatacatcatcttaaataattttaaaattgattcaactcttaaaaccgaaggcgtatttagtagctatatagcaactgctaaggaaaatttacaaaccttaaaagctcaaagtcaaaatgaagaacaagcattaatctttgataaagctttagctattttaaacaatatcactcaaaatgatgattataaactcaatcttgatttaaaatttaaaaacatccctgttagtgactatagcacacagggaatcgacagcatagaaaaactaagcatcaacaatcaagacgccacagaagcgttaaaaattatacttccatttatcatgttttctatgttaatgggcggagcgtcattttaaggatccggctgctaacaaagcccgaaaggaagcgagtgc

SEQ ID NO: 5 - DNA, 33 bp, Artificial Sequence
  forward primer
ccatatgtgcggaaattccatagatgaaaaaac

SEQ ID NO: 6 - DNA, 26 bp, Artificial Sequence
  reverse primer
gggatccttaaaatgacgctccgccc

SEQ ID NO: 7 - protein, 372 aa, Campylobacter jejuni
MKKGIFLSIGIAVLFSACGNSIDEKTVKKYENQLNQTVKQEIASLSQDSGIKIEFSDFKCNADGDFIACLSPNFKTLAKDNNDEYQELFQAKNIKIRSNEIYKGEANTSISIKEYYNDLFKNQKSIQSNLVFEDFKLGEKVVSDINASLFQQDPKIRSFINKLSSDSYTLSFDNSINKQENNYLDNLDIKFYNAKLNFNTNLNINLKEDLLNYLDSKGIKFNTQTLVMDKQAINELLNIANYEQASDFSNTIQKYIILNNFKIDSTLKTEGVFSSYIATAKENLQTLKAQSQNEEQALIFDKALAILNNITQNDDYKLNLDLKFKNIPVSDYSTQGIDSIEKLSINNQDATEALKIILPFIMFSMLMGGASF

SEQ ID NO: 8 - protein, 372 aa, Campylobacter jejuni
MKKGIFLSIGIAVLFSACGNSIDEKTVKKYENQLNQTVKQEIASLSQDSGIKIEFSDFKCNADGDFIACLSPNFKTLAKDNNDEYQELFQAKNIKIRSNEIYKGEANTSISIKEYYNDLFKNQKSIQSNLVFEDFKLGEKVVSDINASLFQQDPKIRSFINKLSSDSYTLSFDNSINKQENNYIDNLDIKFYNAKLNFNTNLNINLKEDLLNYLDSKGIKFNTQTLAMDEQAINELLNIANYEQASDFSNTIQKYIILNNFKIDSTLKTEGVFSSYITTAKENLQTLKTQSQNEEQALIFDKALAILNNITQNDDYKLNLDLKFKNIPVSDYSTQGIDSIEKLSINNQDGTEALKIILPFIMFSMLMGGASF

SEQ ID NO: 9 - protein, 372 aa, Campylobacter jejuni
MKKGIFLSIGIAVLFSACGNSIDEKTVKKYENQLNQTVKQEIASLSQDSGIKIEFSDFKCNADGDFIACLSPNFKTLAKDNNDEYQELFQAKNIKIRSNEIYKGETNTSISIKEYYNDLFKNQKSIQSNLVFEDFKLGEKVVSDINASLFQQDPKISSFINKLSSDSYTLSFDNSINKQENNYLDNLDIKFYNAKLNFNTNLNINLKEDLLNYLDSKGIKFNTQTLAMDEQAINELLNMVNYEQASDFSNTIQKYIILNNFKIDSTLKTEGVFSSYIATAKENLQTLKAQSQNEEQALIFDKALAILNNITQNDDYKLNLDLKFKNIPVSDYSTQGIDSIEKLSINNQDATEALKIILPFIMFSMLMGGASF

SEQ ID NO: 10 - protein, 372 aa, Campylobacter jejuni
MKKGIFLSIGIAVLFSACGNSIDEKTVKKYENQLNQTVKQEIASLSQDSGIKIKFSDFKCNADGDFIACLSPNFKTLAKDNNDEYQELFQAKNIKIRSNEIYKGEANTSISIKEYYNDLFKNQKSIQSNLVFEDFKLGEKVVSDINASLFQQDPKIRSFINKLSSDSYTLSFDNSINKQENNYLDNLDIKFYNAKLNFNTNLNINLKEDLLNYLDSKGIKFNTQTLAMDEQAINELLNIANYEQASNFSNTIQKYIILNNFKIDSTLKTEGVFSSYITTAKENLQTLKTQSQNEEQALIFDKALAILNNITQNDDYKLNLDLKFKNIPVSDYSTQGIDSIEKLSINNQDATEALKIILPFIMFSMLMGGASF

SEQ ID NO: 11 - protein, 372 aa, Campylobacter jejuni
MKKGIFLSIGIAVLFSACGNSIDEKTVKKYENQLNQTVKQEIASLSQDSGIKIEFSDFKCNADGDFIACLSPNFKTLTKDNNDEYQELFQAKNIKIRSNEIYKGEANASISIKEYYNDLFKNQKSIQSNLVFEDFKLGEKVVSDINASLFQQDPKISSFINKLSSDSYTLSFDNSINKQENNYLDNLDIKFYNAKLNFNTNLNINLKEDLLNYLDSKGIKFNTQTLVMDKQAINELLNIANYEQASDFSNTIQKYIILNNFKIDSTLKTEGVFSSYIATAKENLQTLKAQSQNEEQALIFDKALAILNNITQNDDYKLNLDLKFKNIPVSDYSTQGIDSIEKLSINNQDATEALKIILPFIMFSMLMGGASF

SEQ ID NO: 12 - protein, 372 aa, Campylobacter jejuni
MKKGIFLSIGIAVLFSACGNSIDEKTVKKYENQLNQTVKQEIASLSQDSGIKIEFSDFKCNADGDFIACLSPNFKTLTKDNNDEYQELFQAKNIKIRSNEIYKGKANASISIKEYYNDLFKNQKSIQSNLVFEDFKLGEKVVSDINASLFQQDPKISSFINKLSSDSYTLSFDNSINKQENNYLDNLDIKFYNAKLNFNTNLNINLKEDLLNYLDSKGIKFNTQTLVMDKQAINELLNIANYEQASDFSNTIQKYIILNNFKIDSTLKTEGVFSSYIATAKENLQTLKAQSQNEEQALIFDKALAILNNITQNDDYKLNLDLKFKNIPVSDYSTQGIDSIEKLSINNQDATEALKIILPFIMFSMLMGGASF

SEQ ID NO: 13 - protein, 372 aa, Campylobacter jejuni
MKKGIFLSIGIAVLFSACGNSIDEKTVKKYENQLNQTVKQEIASLSQDSGIKIEFSDFKCNADGDFIACLSPNFKTLAKDNNDEYQELFQAKNIKIRSNEIYKGEANTSISIKEYYNDLFKNQKSIQSNLVFEDFKLGEKVVSDINASLFQQDPKISSFINKLSSDSYTLSFDNSINKQENNYLDNLDIKFYNAKLNFNTNLNINLKEDLLNYLDSKGIKFNTQTLAMDEQAINELLNMVNYEQASDFSNTIQKYIILNNFKIDSTLKTEGVFSSYIATAKENLQTLKAQSQNEEQALIFDKALAILNNITQNDDYKLNLDLKFKNIPVSDYSTQGIDSIEKLSINNQDATEALKIILPFIMFSMLMGGASF

SEQ ID NO: 14 - protein, 372 aa, Campylobacter jejuni
MKKGIFLSIGIAVLFSACGNSIDEKTVKKYENQLNQTVKQEIASLSQDSGIKIEFSDFKCNADGDFIACLSPNFKTLAKDNNDEYQELFQAKNIKIRSNEIYKGEANTSISIKEYYNDLFKNQKSIQSNLVFEDFKLGEKVVSDINASLFQQDPKISSFINKLSSDSYTLSFDNSINKQENNYIDNLDIKFYNAKLNFNTNLNINLKEDLLNYLDSKGIKFNTQTLAMDEQAINELLNIANYEQASDFSNTIQKYIILNNFKIDSTLKTEGVFSSYITTAKENLQTLKTQSQNEEQALIFDKALAILNNITQNDDYKLNLDLKFKNIPVSDYSTQGIDSIEKLNINNQDATEALKIILPFIMFSMLMGGASF

SEQ ID NO: 15 - protein, 372 aa, Campylobacter jejuni
MKKGIFLSIGIAVLFSACGNSIDEKTVKKYENQLNQTVKQEIASLSQDSGIKIEFSDFKCNADGDFIACLSSNFKTLAKDNKDEYQELFQAKNIKIRSNEIYKGEANASISIKEYYNDLFKNQKSIQSNLVFENFKLGEKVVSDINASLFQQDPKISSFINKLSSDSYTLSFDNSINKQENNYLDNLDIKFYNAKLNFNTNLNINLKEDLLNYLDSKGIKFNTQTLAMDEQAINELLNMANYEQASDFSNTIQKYIILNNFKIDSTLKTEGVFSSYIATAKENLQTLKAQSQNEEQALIFDKALAILNNITQNDDYKLNLDLKFKNIPVSDYSTQGIDSIEKLSINNQDATEALKIILPFIMFSMLMGGASF

SEQ ID NO: 16 - protein, 280 aa, Campylobacter jejuni
MKKGIFLSIGIAVLFSACGNSIDEKTVKKYENQLNQTVKQEIASLSQDSGIKIEFSDFKCNADGDFIACLSPNFKTLAKDNNDEYQELFQAKNIKIRSNEIYKGEANTSISIKEYYNDLFKNQKSIQSNLVFEDFKLGEKVVSDINASLFQQDPKIRSFINKLSSDSYTLSFDNSINKQENNYLDNLDIKFYNAKLNFNTNLNINLKEDLLNYLDSKGIKFNTQTLAMDEQAINELLNIANYEQASNFSNTIQKYIILNNFKIDSTLKTEGVFSSYITTA